(19) United States
(12) Patent Application Publication
(10) Pub. No.: US 2020/0251215 A1
(43) Pub. Date: Aug. 6, 2020

(54) INFORMATION PROCESSING DEVICE, DIAGNOSIS SUPPORT SYSTEM, AND RECORDING MEDIUM

(71) Applicant: KONICA MINOLTA, INC., Tokyo (JP)

(72) Inventors: Eri NEMOTO, Tokyo (JP); Satoshi KASAI, Tokyo (JP); Hitoshi FUTAMURA, Tokyo (JP)

(21) Appl. No.: 16/778,765

(22) Filed: Jan. 31, 2020

(30) Foreign Application Priority Data
Feb. 1, 2019 (JP) 2019-016588

(51) Int. Cl. G16H 50/20; G16H 10/60; G16H 50/70; G16H 30/20; G16H 30/40

(52) U.S. Cl. CPC G16H 50/20; G16H 10/60; G16H 30/40; G16H 30/20; G16H 50/70

(57) ABSTRACT

An information processing device includes a hardware processor that (i) acquires one or more pieces of examination data concerning a patient targeted to be diagnosed, (ii) specifies candidates for a name of a disease by which the patient targeted to be diagnosed may be affected and a subsequent examination to be executed for determining the name of the disease of the patient targeted to be diagnosed based on the examination data as acquired, and (iii) outputs the candidates for the name of the disease and the subsequent examination as specified.

### CROSS-REFERENCE TO RELATED APPLICATIONS

The present application claims priority under 35 U.S.C. § 119 to Japanese Patent Application No. 2019-016588 filed on Feb. 1, 2019, the entire contents of which being incorporated herein by reference.

### BACKGROUND

### Technological Field

The present invention relates to an information processing device, a diagnosis support system, and a recording medium.

### Description of the Related Art

When diagnosing a patient and determining an approach to treatment, clinicians generally need to make many decisions as to which examination is to be performed for the patient, what kind of information is to be collected, how a large amount of collected information is to be interpreted, and the like. If examinations are blindly performed for the patient with the intention of eliminating unreliability of diagnosis, the patient may have to bear an increased risk and an increased medical expense. Thus, clinicians need to optimize examination data to be acquired and their combination for each patient, rather than merely acquiring a large amount of data.

Clinical decision making usually depends on experiences and knowledge of clinicians. Thus, there is proposed a system of providing clinicians with information about previous cases that have been diagnosed in the past and are similar to a subject case as knowledge to support clinical decision making.

For example, there is proposed a system of extracting features from an original image, and retrieving similar cases in other modalities from a feature relationship between images of a plurality of modalities (see JP2011-508917T).

There is also proposed a similar image retrieval device that retrieves similar images having image properties similar to those of an input image, and calculates a statistical index such as an execution rate concerning an image diagnosis per imaging modality on the basis of diagnostic information associated with the similar images (see JP2007-275440A).

### SUMMARY

However, the name of a disease of a patient is not presented in the above related art, and thus, information effective for clinicians to consider future examinations has been demanded.

For example, in the technology described in JP2011-508917T, clinicians may face difficulty in making determinations even if a plurality of cases are presented.

Also in the technology described in JP2007-275440A, it is difficult to determine whether an examination performed by each modality is effective for a diagnosis by merely presenting a statistical index per imaging modality.

The present invention was made in view of the above-described problems in the related art, and has an object to present subsequent examinations that should be executed next together with information with which it can be determined whether the subsequent examinations are effective for a diagnosis.

To achieve at least one of the abovementioned objects, according to an aspect of the present invention, an information processing device includes:

a hardware processor that

acquires one or more pieces of examination data concerning a patient targeted to be diagnosed,

specifies candidates for a name of a disease by which the patient targeted to be diagnosed may be affected and a subsequent examination to be executed for determining the name of the disease of the patient targeted to be diagnosed based on the examination data as acquired, and

outputs the candidates for the name of the disease and the subsequent examination as specified.

According to another aspect of the present invention, a diagnosis support system includes:

a hardware processor that

acquires one or more pieces of examination data concerning a patient targeted to be diagnosed,

specifies candidates for a name of a disease by which the patient targeted to be diagnosed may be affected and a subsequent examination to be executed for determining the name of the disease of the patient targeted to be diagnosed based on the examination data as acquired, and

outputs the candidates for the name of the disease and the subsequent examination as specified.

According to still another aspect of the present invention, a non-transitory recording medium stores a computer readable program that causes a computer to:

acquire one or more pieces of examination data concerning a patient targeted to be diagnosed;

specify candidates for a name of a disease by which the patient targeted to be diagnosed may be affected and a subsequent examination to be executed for determining the name of the disease of the patient targeted to be diagnosed based on the examination data as acquired; and

output the candidates for the name of the disease and the subsequent examination as specified.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention.

FIG. 1 is a system configuration diagram of a diagnosis support system in a first embodiment of the present invention.

FIG. 2 is an example of a management table.

FIG. 3 is a flowchart showing discriminator generation processing.

FIG. 4 is an example of an invalid data designation window.

FIG. 5 is a flowchart showing first diagnosis support processing.

FIG. 6 is a presentation example of candidates for the name of a disease and subsequent examinations.

FIG. 7 is a presentation example of candidates for the name of a disease, confidence rates, subsequent examinations, and a diagnosis determination rate.

FIG. 8 is a flowchart showing recommended examination item preparation processing in a second embodiment.

FIG. 9 is a diagram for describing a method of deciding recommended examination items.

FIG. 10 is a flowchart showing second diagnosis support processing.

FIG. 11 is a diagram for describing a method of specifying subsequent examinations in a case where two candidates for the name of a disease have been specified.

FIG. 12 is a diagram for describing how to obtain a true positive rate in a modification of the second embodiment.

FIG. 13 is a diagram for describing a method of specifying subsequent examinations based on a true positive rate.

### DETAILED DESCRIPTION OF THE EMBODIMENTS

Hereinafter, one or more embodiments of the present invention will be described with reference to the drawings. However, the scope of the invention is not limited to the disclosed embodiments.

### First Embodiment

A first embodiment of the present invention will be described first. However, the scope of the invention is not limited to illustrated examples.

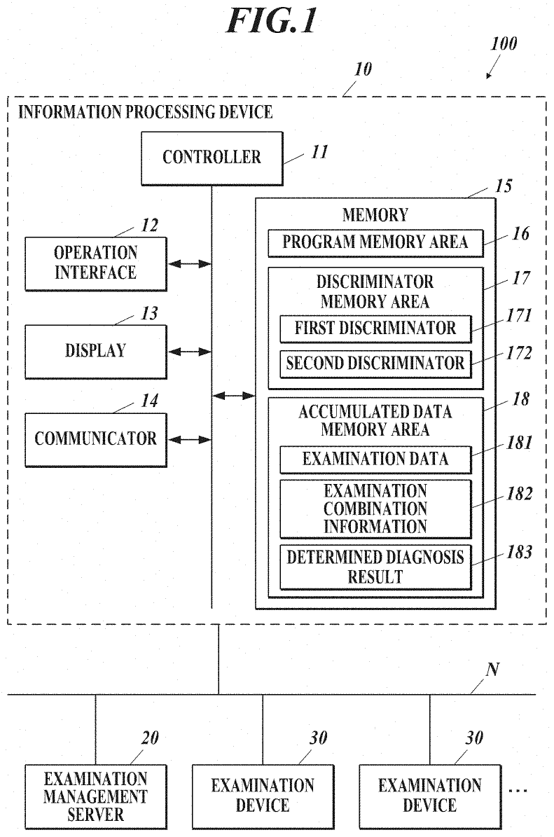

A system configuration of a diagnosis support system 100 in the first embodiment is shown in FIG. 1.

As shown in FIG. 1, the diagnosis support system 100 is configured to include an information processing device 10, an examination management server 20, and examination devices 30. The information processing device 10, the examination management server 20, and the examination devices 30 are connected so as to be capable of performing data communication via a communication network N. The information processing device 10, the examination management server 20, and the examination devices 30 are provided in the same medical facility, for example.

The information processing device 10 is a computer device such as a PC (personal computer) to be used by clinicians who belong to the medical facility. The information processing device 10 is used when considering the next examination (a subsequent examination) for a patient having undergone one or more examinations, and the like.

The examination management server 20 manages information concerning examinations executed for patients. The examination management server 20 holds, for each patient, items of examinations executed for the patient, examination data (examination results), and the name of a disease (determined diagnosis result) diagnosed for the patient in association with each other. The examination management server 20 provides various types of information for the information processing device 10 in response to a request made by the information processing device 10.

The examination management server 20 also manages a reservation status of the examination devices 30. The examination management server 20 holds reserved dates and times for each of the examination devices 30.

The examination devices 30 are various modalities, examination devices for examinations other than an imaging examination, or the like.

The information processing device 10 is configured to include a controller 11 (hardware processor), an operation interface 12, a display 13, a communicator 14, a memory 15, and the like, and the respective components are connected via a bus.

The controller 11 is composed of a central processing unit (CPU), a random access memory (RAM), and the like, and exerts centralized control over processing operations of the respective components of the information processing device 10. Specifically, the CPU reads out various processing programs held in a program memory area 16 of the memory 15 for expansion to the RAM, and executes various types of processing in conjunction with the programs.

The operation interface 12 is configured to include a keyboard including a cursor key, character input keys, various functional keys, and the like as well as a pointing device such as a mouse, and outputs an operation signal input through a key operation on the keyboard or a mouse operation to the controller 11. Alternatively, the operation interface 12 may be composed of a touch panel laminated on the display 13, so that an operation signal in accordance with the position of a touch operation made by an operator with a finger or the like is output to the controller 11.

The display 13 is configured to include a monitor such as a liquid crystal display (LCD), and displays various windows in accordance with an instruction of a display signal input from the controller 11.

The communicator 14 is composed of a network interface or the like, and transmits/receives data to/from external equipment connected via the communication network N such as a local area network (LAN), a wide area network (WAN), or the Internet.

The memory 15 is composed of a hard disk drive (HDD), a nonvolatile semiconductor memory, or the like, and holds various types of data. The memory 15 has the program memory area 16, a discriminator memory area 17, and an accumulated data memory area 18.

Various processing programs to be executed in the information processing device 10 are held in the program memory area 16.

A first discriminator 171 and a second discriminator 172 are held in the discriminator memory area 17.

The first discriminator 171 is obtained by causing machine learning to be performed using examination data as an input and the name of a disease of the patient having undergone examinations related to the examination data as an output.

The second discriminator 172 is obtained by causing machine learning to be performed using examination data as an input and a combination of examinations having been executed for the patient undergone examinations related to the examination data and having been effective in determining the name of a disease as an output.

Examination data to be used in learning for the discriminators (the first discriminator 171 and the second discriminator 172) and examination data to be input to the discriminators when utilizing the discriminators include (i) medical images, (ii) biological examination results, (iii) clinical information, and the like.

### (i) Medical Image

As medical images, images captured by various imaging modalities such as mammography, ultrasound, CT, MRI, PET, plain x-ray, and the like are used. Pathological images such as HE, histochemically-stained, and immunohistochemically-stained images are also used as medical images.

### (ii) Biological Examination Result

As a biological examination result, a tumor marker analysis, an amino acid analysis, or a genetic analysis executed on a blood or biopsy (surgical) sample is used. Examples of the genetic analysis include gene pathway activation, noncoding RNA, multiple RNAs, single nucleotide polymorphism, copy number polymorphism, epigenetic polymorphism, and the like obtained through a microarray analysis, polymerase chain reaction (PCR), gene (DNA/RNA) sequence analysis, and the like.

### (iii) Clinical Information

Clinical information includes various types of medical related information accumulated in a medical facility, such as patient information contained in electronic chart information, computer diagnosis support system (CAD) information, receipt information, and the like, information about diagnoses made by clinicians including image reading reports, and the like.

Examination data 181, examination combination information 182, and a determined diagnosis result 183 are held in the accumulated data memory area 18.

The examination data 181 is information indicating a result of an examination executed for each patient. The examination data 181 includes one or more of a medical image, a biological examination result, and clinical information. A patient targeted for examination, an examination item, and the like are added to the examination data 181 as additional information. That is, a patient and an examination item related to the examination data 181 can be specified with reference to the additional information in the examination data 181.

The examination combination information 182 is information indicating a combination of examinations executed for each patient.

The determined diagnosis result 183 is information indicating the name of a disease (the name of a pathological tissue or the like) diagnosed for each patient.

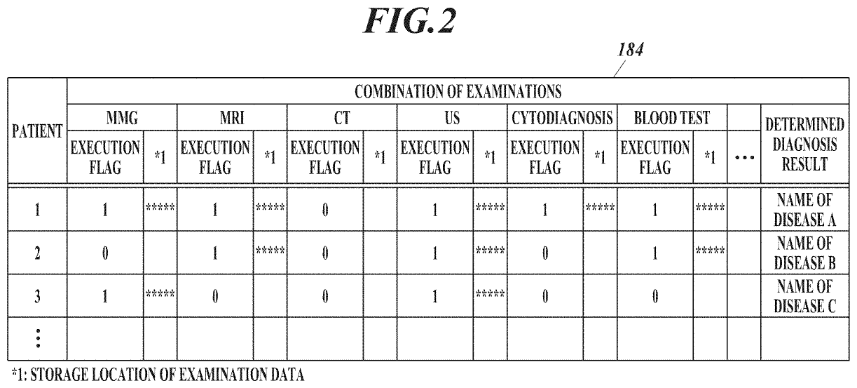

An example of a management table 184 held in the accumulated data memory area 18 is shown in FIG. 2. The management table 184 indicates a correspondence relationship between a patient, the examination combination information 182, the examination data 181, and the determined diagnosis result 183.

In the management table 184, for each patient, an execution flag (1: executed, 0: unexecuted) indicating whether each examination (such as MMG, MRI, CT, US, cytodiagnosis, or blood test) has been executed for the patient, a storage place of examination data corresponding to examinations having been executed, and a determined diagnosis result indicating the name of a disease determined for the patient are associated with each other. Examination items registered as examinations having been executed for each patient only include examinations having been effective in determining the name of a disease. That is, in the accumulated data memory area 18 in the memory 15, for each patient, one or more examination items only including examinations having been executed for each patient and effective in determining the name of a disease, the examination data 181 corresponding to the examination items, and the determined diagnosis result 183 indicating the name of a disease determined for the patient are held in association with each other. Examinations executed for each patient (the examination combination information 182), the examination data 181, and the determined diagnosis result 183 can be specified with reference to the management table 184.

In the information processing device 10, the controller 11 acquires one or more pieces of examination data concerning a patient targeted to be diagnosed. For example, the controller 11 acquires examination data about examinations executed for the patient targeted to be diagnosed from the examination management server 20 via the communicator 14.

The controller 11 specifies candidates for the name of a disease by which the patient targeted to be diagnosed may be affected and subsequent examinations to be executed for determining the name of a disease of the patient targeted to be diagnosed on the basis of the acquired examination data. Candidates for the name of a disease are the names of pathological tissues acquired when determining a diagnosis.

Specifically, the controller 11 specifies candidates for the name of a disease of the patient targeted to be diagnosed from examination data about the patient targeted to be diagnosed using the first discriminator 171 having undergone machine learning in advance using examination data about examinations having been effective in determining the name of a disease as an input and the name of the disease of the patient having undergone examinations related to the examination data as an output.

The controller 11 also specifies subsequent examinations to be executed for the patient targeted to be diagnosed from examination data about the patient targeted to be diagnosed using the second discriminator 172 having undergone machine learning in advance using examination data about examinations having been effective in determining the name of a disease as an input and a combination of examinations having been executed for the patient undergone examinations related to the examination data and having been effective in determining the name of a disease as an output.

The controller 11 outputs the candidates for the name of a disease and the subsequent examinations as specified. Specifically, the controller 11 causes the candidates for the name of a disease and the subsequent examinations as specified to be displayed on the display 13 (display device).

In a case where there are two or more candidates for the name of a disease, the controller 11 may output a confidence rate together with each of the candidates for the name of a disease. The confidence rate is of the name of a pathological tissue (prediction result) specified as a candidate for the name of a disease.

The controller 11 may also output a diagnosis determination rate in a case where subsequent examinations are performed. The diagnosis determination rate is a rate at which, in a case of executing an examination, the name of a disease is determined after the examination.

Examinations included in the subsequent examinations include one or more of an imaging modality, an imaging technique, a site to be imaged, the type of a biological examination, and examination items.

In a case of an imaging examination by means of an imaging modality, an imaging sequence and imaging conditions are included in an examination order. The imaging sequence includes ultrasound (A, B, M, color doppler, power doppler, wideband doppler), CT (CT, CECT), MRI (T1, T2, DWI, FLAIR, SWI, MRA, BPAS, . . . ), FDG-PET, and the like. The imaging conditions include an imaging direction, a dose, a US technique (in which direction and in what manner ultrasound is emitted), and the like.

The biological examination includes cytodiagnosis, tissue diagnosis, blood test, and the like.

Items of biological examinations include a tumor marker analysis, an amino acid analysis, a genetic analysis, and the like. Examples of the genetic analysis include target markers such as gene pathway activation, noncoding RNA, multiple RNAs, single nucleotide polymorphism, copy number polymorphism, and epigenetic polymorphism obtained through a microarray analysis, polymerase chain reaction (PCR), gene (DNA/RNA) sequence analysis, and the like.

An examination method (imaging examination/biological examination), period, interval, and the like are also included in an order of subsequent examinations as an order concerning pre-surgical treatment/post-surgical follow-up.

Next, an operation in the information processing device 10 of the first embodiment will be described.

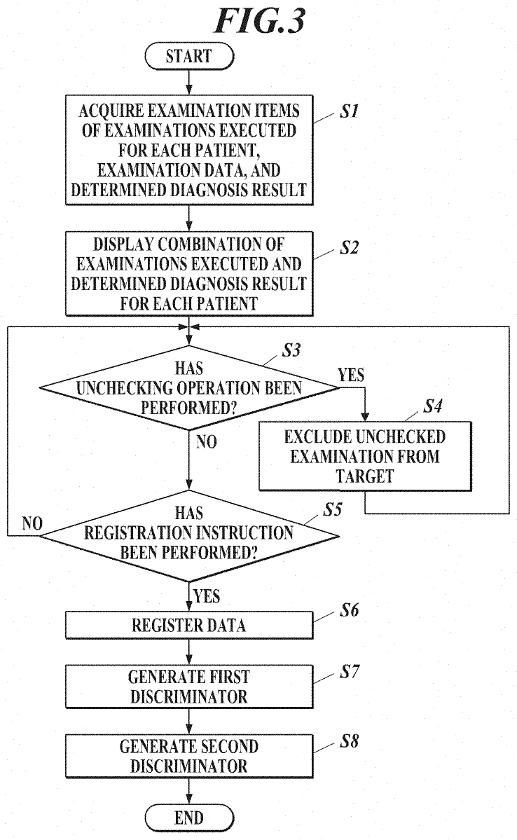

FIG. 3 is a flowchart showing discriminator generation processing executed by the information processing device 10. The discriminator generation processing is executed when generating discriminators (the first discriminator 171 and the second discriminator 172), and is executed in advance before diagnosing a patient. This processing is achieved by software processing through cooperation between the controller 11 and a program held in the memory 15.

First, the controller 11 acquires examination items of examinations executed for each patient, examination data corresponding to each examination item, and a determined diagnosis result for each patient from the examination management server 20 via the communicator 14 (step S1).

Next, the controller 11 causes a combination of examinations having been executed and the determined diagnosis result to be displayed on the display 13 for each patient (step S2).

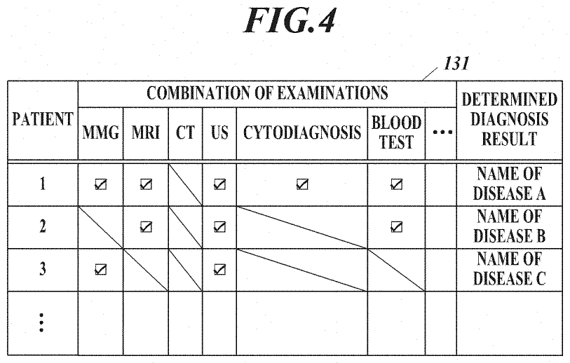

An example of an invalid data designation window 131 displayed on the display 13 is shown in FIG. 4.

On the invalid data designation window 131, a combination of examinations executed for the patient and the determined diagnosis result diagnosed for the patient are displayed in association with each other for each patient. In the display area of a combination of examinations, check marks are displayed for examinations executed a patient. For example, “MMG”, “MRI”, “US”, “cytodiagnosis”, and “blood test” have been executed for a “patient 1”, and “CT” has not been executed.

Considering that the determined diagnosis result of the “patient 1” is a “name of disease A”, a clinician leaves checks for examinations having been effective in diagnosing the “name of disease A”, and unchecks examinations having been ineffective in diagnosing the “name of disease A” through an operation via the operation interface 12. In this manner, the clinician shall leave only examinations having been effective in determining the name of a disease as information.

Next, the controller 11 determines whether an unchecking operation has been executed on any examination via the operation interface 12 on the invalid data designation window 131 displayed on the display 13 (step S3). In a case where an unchecking operation has been executed on any examination (YES in step S3), the controller 11 deletes the check mark corresponding to the relevant examination on the invalid data designation window 131, and excludes the unchecked examination from the target of data to be accumulated (step S4). That is, the controller 11 excludes an examination not contributing to the determination of the name of a disease from a combination of examinations for guiding a determined diagnosis result. A return is made to step S3 after step S4, and the process is repeated.

In a case where an unchecking operation has not been executed on an examination in step S3 (NO in step S3), the controller 11 determines whether an instruction to register data acquired from the examination management server 20 has been performed via the operation interface 12 (step S5). In a case where an instruction to register data acquired from the examination management server 20 has not been performed (NO in step S5), a return is made to step S3, and the process is repeated.

In step S5, in a case where an instruction to register data acquired from the examination management server 20 has been performed (YES in step S5), the controller 11 registers the data acquired from the examination management server 20 in the memory 15 (step S6).

Specifically, the controller 11 causes only the examination data 181 corresponding to a checked examination on the invalid data designation window 131 to be held in the accumulated data memory area 18.

The controller 11 also sets, for each patient, an execution flag of a checked examination (an examination having been executed for the patient and effective in determining the name of a disease) as “1” in the management table 184 (see FIG. 2), and stores a storage location of the examination data 181 corresponding to each examination and the determined diagnosis result 183 indicating the name of a disease determined for the patient in association with each other.

In this manner, the examination combination information 182, the examination data 181, and the determined diagnosis result 183 for each patient are stored in the memory 15 in association with each other.

Next, for each patient whose data is held in the accumulated data memory area 18 in the memory 15, the controller 11 causes machine learning to be performed using the examination data 181 about one or more examinations having been effective in determining the name of a disease of the patient as an input and the name of a disease of the patient (the determined diagnosis result 183) as an output, to generate the first discriminator 171 (step S7). As the number of pieces of the examination data 181 input to the first discriminator 171 increases, the accuracy of predicting the name of a disease to be output increases. The controller 11 also generates the first discriminator 171 such that the confidence rate of the name of a disease is also output together when outputting the name of a disease of the patient using the examination data 181 as an input. The controller 11 causes the generated first discriminator 171 to be held in the discriminator memory area 17 in the memory 15.

Next, for each patient whose data is held in the accumulated data memory area 18 in the memory 15, the controller 11 causes machine learning to be performed using the examination data 181 about examinations having been effective in determining the name of a disease of the patient as an input and a combination of examinations having been executed for the patient undergone examinations related to the examination data 181 and effective in determining the name of a disease as an output, to generate the second discriminator 172 (step S8). Herein, the controller 11 generates the second discriminator 172 such that the diagnosis determination rate in a case where each examination included in the combination is executed is also output together when outputting a combination of examinations using the examination data 181 as an input. The controller 11 causes the generated second discriminator 172 to be held in the discriminator memory area 17 in the memory 15.

The discriminator generation processing is now terminated.

The first discriminator 171 and the second discriminator 172 are composed of various network models (such as AlexNet and GoogleNet) for random forest, decision tree, support vector machine (SVM), and deep learning. The first discriminator 171 is obtained by performing learning associating the determined diagnosis result 183 with the examination data 181 as a correct answer label. The second discriminator 172 is obtained by performing learning associating the examination combination information 182 with the examination data 181 as a correct answer label. Multi-label learning may be performed by associating two types of labels of the determined diagnosis result 183 and the examination combination information 182 with the examination data 181 as correct answer labels. The approach for machine learning and a network used in deep learning may be made selectable arbitrarily, or may be fixed.

In the discriminator generation processing, only information added after the latest processing may be acquired to update the first discriminator 171 and the second discriminator 172, instead of acquiring all the pieces of information managed by the examination management server 20.

In the discriminator generation processing, processing from step S1 to step S6 (until data is registered) and processing in steps S7 and S8 (generation of discriminators) may be executed at different timings. Alternatively, the processing from step S1 to step S6 may be executed each time a determined diagnosis result is generated, and the processing in steps S7 and S8 may be executed at a timing when data sufficient for generating the discriminators is accumulated.

When generating the first discriminator 171 and the second discriminator 172, features may be extracted from the examination data 181, and the first discriminator 171 and the second discriminator 172 may be generated using the extracted features as an input.

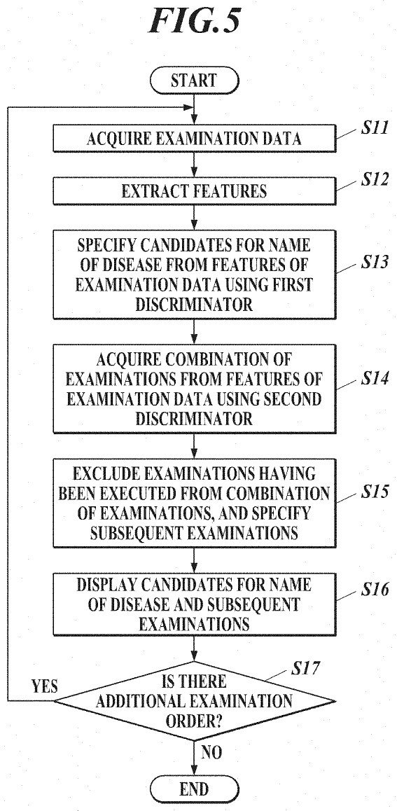

FIG. 5 is a flowchart showing first diagnosis support processing executed by the information processing device 10. This processing is executed when considering the name of a disease of a patient targeted to be diagnosed and subsequent examinations, and is achieved by software processing through cooperation between the controller 11 and a program held in the memory 15.

First, the controller 11 acquires one or more pieces of examination data (examination result) concerning the patient targeted to be diagnosed (step S11). Specifically, the controller 11 acquires examination data about examinations executed for the patient targeted to be diagnosed from the examination management server 20 via the communicator 14. The controller 11 acquires one or more pieces of data among a medical image, a biological examination result, and clinical information.

The controller 11 may acquire examination data about the patient targeted to be diagnosed held in advance in the memory 15 of the information processing device 10 or an external device.

Next, the controller 11 extracts features from the acquired one or more pieces of examination data (step S12). For example, in a case where an image has been acquired as examination data, the controller 11 extracts various image features such as features of the density, shape, texture, and wavelet transformation-based definition of a lesion.

Next, the controller 11 specifies candidates for the name of a disease by which the patient targeted to be diagnosed may be affected from the features of examination data using the first discriminator 171 held in the discriminator memory area 17 in the memory 15 (step S13). The controller 11 also acquires the confidence rates of the specified candidates for the name of a disease from the features of examination data using the first discriminator 171.

In step S13, the features of examination data are input to the first discriminator 171, but in the case of deep learning, the examination data itself is used as an input to the first discriminator 171 without independently extracting the features.

Next, the controller 11 acquires a combination of examinations having been effective in determining the name of a disease from the features of examination data using the second discriminator 172 held in the discriminator memory area 17 in the memory 15 (step S14). The controller 11 also acquires the diagnosis determination rate in a case of executing each examination included in the acquired combination of examinations from the features of examination data using the second discriminator 172.

In step S14, the features of examination data are input to the second discriminator 172, but in the case of deep learning, the examination data itself is used as an input to the second discriminator 172 without independently extracting the features.

Next, the controller 11 excludes examinations having been executed from the combination of examinations as acquired, and specifies subsequent examinations to be executed for determining the name of a disease of the patient targeted to be diagnosed (step S15).

Next, the controller 11 causes the candidates for the name of a disease and the subsequent examinations as specified to be displayed on the display 13 (step S16). The clinician makes an additional examination order if there are examinations that should be executed among the subsequent examinations displayed on the display 13 while referring to the candidates for the name of a disease displayed on the display 13.

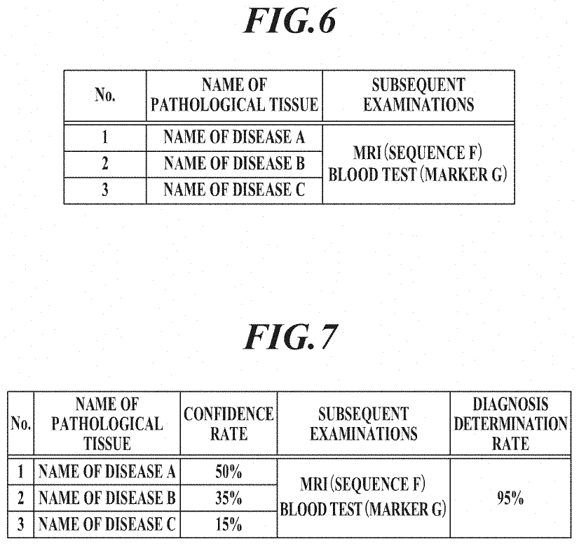

A presentation example of candidates for the name of a disease (the name of a pathological tissue) and subsequent examinations are shown in FIG. 6. Herein, a blood test result (a marker E) and an MMG image are used as examination data to be input to the first discriminator 171 and the second discriminator 172, and the names of pathological tissues (candidates for the name of a disease) are output from the first discriminator 171, and examinations are output from the second discriminator 172.

Furthermore, the controller 11 can also cause the confidence rate to be displayed together for each of the candidates for the name of a disease, and can also cause the diagnosis determination rate in a case of executing the presented subsequent examinations to be displayed together.

A presentation example of candidates for the name of a disease (the name of a pathological tissue), confidence rates, subsequent examinations, and a diagnosis determination rate is shown in FIG. 7. Herein, a blood test result (the marker E) and an MMG image are used as examination data to be input to the first discriminator 171 and the second discriminator 172, and the names of pathological tissues (candidates for the name of a disease) and the confidence rates are output from the first discriminator 171, and examinations and a diagnosis determination rate are output from the second discriminator 172. Specifically, as a result of the blood test (the marker E) and MMG, the patient targeted to be diagnosed is determined as having the “name of disease A” at a confidence rate of “50%”, a “name of disease B” at a confidence rate of “35%”, and a “name of disease C” at a confidence rate of “15%”. As subsequent examinations, “MRI (sequence F)” and the “blood test (marker G)” are presented, and the rate at which a diagnosis of any of the “name of disease A”, the “name of disease B”, and the “name of disease C” is determined by executing these examinations is “95%”.

Weights may be set for subsequent examinations to be presented on the basis of the confidence rates of the names of pathological tissues to decide a combination of examination items effective for a diagnosis.

The clinician can arbitrarily designate examinations to be executed among the presented subsequent examinations. For example, the controller 11 receives an operation from a user interface that designates examinations to be executed or examinations not to be executed, and causes the designated information to be held in the memory 15. The clinician can make an arbitrary selection as to which examination is to be given priority.

Next, the controller 11 determines whether an additional examination order has been instructed by an operation made via the operation interface 12 (step S17).

In a case where an additional examination order has been instructed (YES in step S17), examinations related to the examination order are executed for the patient targeted to be diagnosed, and then, a return is made to step S11. Specifically, the controller 11 acquires examination data about the examinations related to the additional examination order (step S11), adds the acquired examination data, and specifies candidates for the name of a disease (step S13) and specifies subsequent examinations (steps S14 and S15).

In a case where an additional examination order is not instructed in step S17 (NO in step S17), the first diagnosis support processing is terminated.

As described above, in accordance with the first embodiment, candidates for the name of a disease and subsequent examinations are specified on the basis of examination data about the patient targeted to be diagnosed. Thus, subsequent examinations that should be executed next can be presented together with information (candidates for the name of a disease) with which it can be determined whether the subsequent examinations are effective for a diagnosis. By performing only examinations necessary for the patient, a burden and a medical expense to be borne by the patient can be reduced.

Specifically, candidates for the name of a disease of the patient targeted to be diagnosed can be specified from examination data about the patient targeted to be diagnosed using the first discriminator 171 having undergone machine learning in advance.

Subsequent examinations to be executed for the patient targeted to be diagnosed can also be specified from the examination data about the patient targeted to be diagnosed using the second discriminator 172 having undergone machine learning in advance.

By outputting the confidence rate together for each of the candidates for the name of a disease, information effective for a clinician in determining the name of a disease of the patient targeted to be diagnosed, and selecting subsequent examinations is obtained.

By outputting the diagnosis determination rate in a case of executing subsequent examinations together with the subsequent examinations, information effective for the clinician in selecting the subsequent examinations is obtained.

### Second Embodiment

Next, a second embodiment to which the present invention has been applied will be described.

Since a diagnosis support system in the second embodiment has a configuration similar to that of the diagnosis support system 100 described in the first embodiment, FIG. 1 is employed, and description of the same components will be omitted. A configuration and processing specific to the second embodiment will be described.

In the second embodiment, subsequent examinations are specified using statistical values or the like instead of the second discriminator 172.

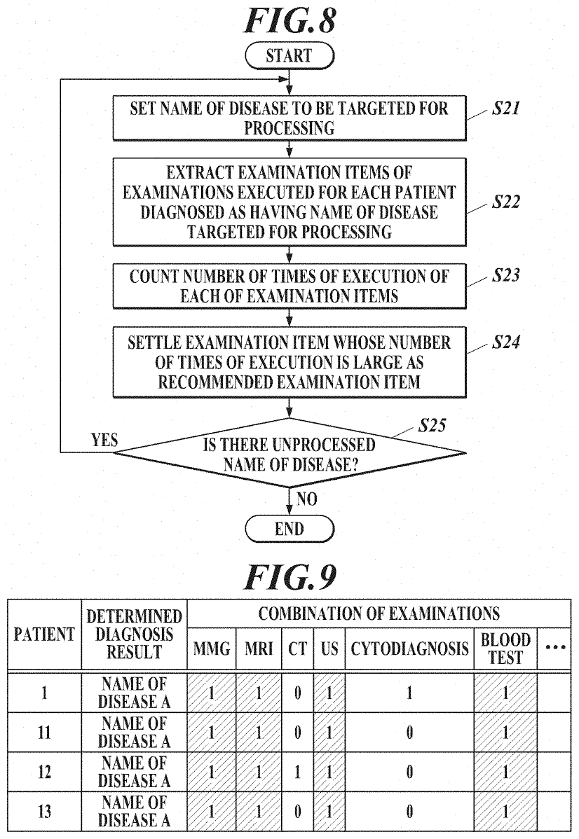

The controller 11 of the information processing device 10 specifies subsequent examinations to be executed for the patient targeted to be diagnosed using a statistical analysis on the basis of a combination of examinations executed for determining the name of a disease, having been accumulated in advance for the specified candidates for the name of a disease. Specifically, in recommended examination item preparation processing (see FIG. 8), the controller 11 obtains recommended examination items that should be executed as subsequent examinations for each name of a disease, and with reference to this correspondence relationship, specifies subsequent examinations suitable for the specified candidates for the name of a disease.

Next, an operation in the information processing device 10 of the second embodiment will be described.

In the second embodiment, the discriminator generation processing shown in FIG. 3 is also executed in advance before diagnosing a patient. However, the second discriminator 172 is not used in the second embodiment, and thus, the processing in step S8 is not executed.

FIG. 8 is a flowchart showing the recommended examination item preparation processing executed by the information processing device 10. The recommended examination item preparation processing is for statistically obtaining an examination item having a high execution rate for each name of a disease with reference to the management table 184 (see FIG. 2) held in the memory 15, and is executed in advance before diagnosing a patient. This processing is achieved by software processing through cooperation between the controller 11 and a program held in the memory 15.

First, the controller 11 refers to a “determined diagnosis result” field of the management table 184 held in the memory 15, and sets the name of a disease to be targeted for processing (determined diagnosis result) (step S21).

Next, the controller 11 extracts examination items of examinations executed for each patient diagnosed as having the name of a disease targeted for processing from the management table 184 (step S22).

A result of extracting the execution flag of each examination from the management table 184 for each patient whose “determined diagnosis result” is the “name of disease A” is shown in FIG. 9. In FIG. 9, an examination item whose execution flag is “1” is an examination item of an examination having been executed for a patient and effective in determining the name of a disease.

Next, the controller 11 counts the number of times of execution of each of the extracted examination items (step S23). Specifically, in FIG. 9, the number of execution flags “1” are summed up for each of the examination items. That is, for each of the examination items, the controller 11 calculates the number of patients for whom the examination has been effective in determining the name of a disease among patients diagnosed as having the name of a disease targeted for processing.

Next, the controller 11 settles an examination item whose number of times of execution is large as a recommended examination item (step S24). Whether “the number of times of execution is large” may be determined according to whether the number of times of execution is larger than a predetermined number of times, or may be determined according to whether a ratio of the number of times of execution (execution rate) to the number of patients diagnosed as having the name of a disease targeted for processing is larger than a predetermined value. The controller 11 causes the name of a disease targeted for processing and recommended examination items to be held in the memory 15 in association with each other. For example, in FIG. 9, since examinations of “MMG”, “MRI”, “US”, and “blood test” have been executed for all of four patients diagnosed as having the “name of disease A”, these examination items are set as recommended examination items, and are employed as effective examinations for deciding the name of disease A.

Next, the controller 11 determines whether there is an unprocessed name of a disease (determined diagnosis result) in accumulated data managed in the management table 184 (step S25). In a case where there is an unprocessed name of a disease (YES in step S25), processing is repeated in step S21 targeting at another name of a disease.

In step S25, in a case where processing has been terminated for all the names of diseases (determined diagnosis results) in the accumulated data managed in the management table 184 (NO in step S25), the recommended examination item preparation processing is terminated.

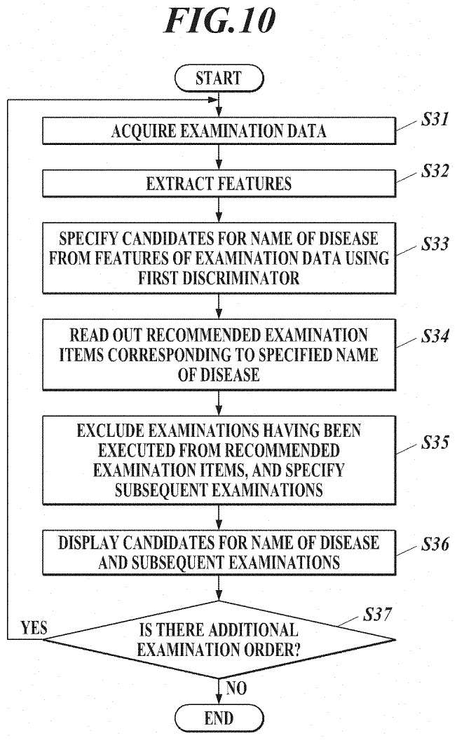

FIG. 10 is a flowchart showing second diagnosis support processing executed by the information processing device 10. This processing is performed when considering the name of a disease and subsequent examinations for a patient targeted to be diagnosed, and is achieved by software processing through cooperation between the controller 11 and a program held in the memory 15.

Since processing from step S31 to step S33 is similar to the processing from step S11 to step S13 in the first diagnosis support processing (see FIG. 5), description will be omitted.

Next, the controller 11 reads out recommended examination items associated with the name of a disease (the name of a disease by which the patient targeted to be diagnosed may be affected) specified in step S33 from the memory 15 (step S34).

Next, since examinations related to examination data (examination data acquired in step S31) input to specify the name of a disease have already been executed, the controller 11 excludes the examinations having been executed from the recommended examination items as read out, and specifies subsequent examinations (step S35).

Since processing from step S36 to step S37 is similar to the processing from step S16 to step S17 in the first diagnosis support processing (see FIG. 5), description will be omitted.

In step S33, in a case where the “name of disease A” and the “name of disease B” are specified as candidates for the name of a disease, and distinction is to be made as to which is the name of a disease, the controller 11 compares recommended examination items determined for each name of a disease, and preferentially extracts examinations common to the “name of disease A” and the “name of disease B” for output as examination items of subsequent examinations.

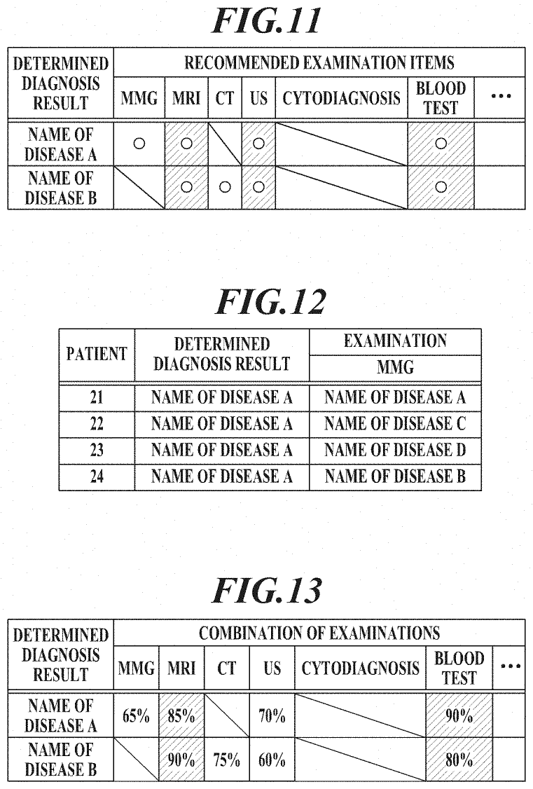

For example, as shown in FIG. 11, in a case where recommended examination items for the “name of disease A” are “MMG”, “MRI”, “US”, and “blood test”, and recommended examination items for the “name of disease B” are “MRI”, “CT”, “US”, and “blood test”, it is determined that subsequent examinations suitable for diagnosing the “name of disease A” and the “name of disease B” are “MRI”, “US”, and “blood test”.

As described above, in accordance with the second embodiment, candidates for the name of a disease and subsequent examinations are specified on the basis of examination data about a patient targeted to be diagnosed. Thus, subsequent examinations that should be executed next can be presented together with information (candidates for the name of a disease) with which it can be determined whether the subsequent examinations are effective for a diagnosis.

Specifically, candidates for the name of a disease of the patient targeted to be diagnosed can be specified from examination data about the patient targeted to be diagnosed using the first discriminator 171 having undergone machine learning in advance.

By obtaining a correspondence relationship between the name of a disease and recommended examination items through a statistical analysis on the basis of a combination of examinations executed for determining the name of a disease, having been accumulated in advance, and using this correspondence relationship, subsequent examinations to be executed for the patient targeted to be diagnosed can be specified.

### Modification

Next, a modification of the second embodiment will be described.

In the modification, for each examination item, an agreement rate (true positive rate) between a diagnosis result based on examination data about the examination item and a determined diagnosis result is further obtained in advance for each name of a disease. The diagnosis result based on examination data is an undetermined provisional diagnosis result determined from a single piece of examination data. A diagnosis result (the name of a disease) based on the examination data 181 shall be held in the memory 15 in association with each piece of the examination data 181.

Diagnosis results (diagnosis results based on examination data) obtained from examination data when executing the examination of “MMG” for four patients finally diagnosed as having the “name of disease A” are shown in FIG. 12. In FIG. 12, by executing the examination of “MMG” for each of the patients, diagnosis results of the “name of disease A”, the “name of disease C”, a “name of disease D”, and the “name of disease B” are obtained respectively. Since there is a patient having been diagnosed as having the “name of disease A” by executing the examination of “MMG” with respect to the determined diagnosis result of the “name of disease A”, the true positive rate of the “name of disease A” in the examination of “MMG” is 1/4=0.25.

In the case where the “name of disease A” and the “name of disease B” are specified as candidates for the name of a disease, and distinction is to be made as to which is the name of a disease, examination whose true positive rate is relatively high is preferentially extracted for either of the names of diseases for output as an examination item of a subsequent examination. The true positive rates of the respective examination items for the “name of disease A” and the “name of disease B” are shown in FIG. 13. For example, in a case of settling examinations whose true positive rate for both the “name of disease A” and the “name of disease B” is more than or equal to 80% as subsequent examinations, “MRI” and “blood test” are output as subsequent examinations. Alternatively, subsequent examinations may be settled giving priority to the true positive rate of the name of a disease having higher severity.

In accordance with the modification, by specifying subsequent examinations giving priority to examinations providing higher true positive rates on the basis of the true positive rate of each examination item, the subsequent examinations can be presented efficiently.

The description in the above embodiments and modification is addressed to an example of the information processing device and diagnosis support system according to the present invention, and this is not a limitation. Each device that constitutes the system, a detailed configuration and a detailed operation of each unit that constitutes the device may also be modified as appropriate within the scope of the present invention.

For example, a configuration or processing specific to each of the above embodiments and modification may be combined.

Although the above embodiments and modification describe the case of displaying candidates for the name of a disease and the subsequent examinations specified from examination data to present the candidates for the name of a disease and the subsequent examinations to a clinician, the candidates for the name of a disease and the subsequent examinations may be output as data in a format that can be utilized by the clinician.

In a case where two or more subsequent examinations are specified, the diagnosis determination rate in a case where the examination is executed may be presented together for each examination item.

In a case where there are a plurality of examination items whose diagnosis determination rates are equivalent, an examination which requires a shorter time may be given higher priority on the basis of the required time of each examination, and an examination having higher priority may be presented as a subsequent examination. Specifically, a value (priority) obtained by adding a weight in accordance with the required time to the diagnosis determination rate of each examination is calculated for ranking, and examinations are displayed as subsequent examinations in accordance with the ranking.

Accordingly, the examination required time can be shortened.

When displaying subsequent examination items, the required time of each examination (or the degree of priority of each examination based on the required time) may be presented together.

With reference to the provided information, the clinician can shorten the examination required time if he/she selects an examination which requires a short examination time. An examination which requires a long time may be omitted at the clinician's discretion (or automatically) even if the diagnosis determination rate is relatively higher than other examination items.

In a case where there are a plurality of examination items whose diagnosis determination rates are equivalent, a less-invasive examination may be given higher priority on the basis of invasiveness of each examination, and an examination having higher priority may be presented as a subsequent examination. The invasiveness refers to the degree of magnitude of a burden on the patient's body. A highly-invasive examination includes an examination involving injection of a contrast agent, biopsy of collecting part of a lesion of a patient with a needle or scalpel, an examination involving exposure, and the like. Specifically, a value (priority) obtained by adding a weight in accordance with the level of invasiveness to the diagnosis determination rate of each examination is calculated for ranking, and examinations are displayed as subsequent examinations in accordance with the ranking.

The burden on the patient can be reduced by presenting examinations which are as less invasive as possible.

When displaying subsequent examination items, the invasiveness of each examination (or the degree of priority of each examination based on the invasiveness) may be presented together.

Accordingly, examinations can be selected in accordance with the physical strength, allergy, and the like of the patient. A highly-invasive examination may be omitted at the clinician's discretion (or automatically) even if the diagnosis determination rate is relatively higher than other examination items.

In a case where two or more subsequent examination items are specified, the order of executing the examinations may be proposed in accordance with the degree of reservation of the examination devices 30. Specifically, the reservation status of each of the examination devices 30 is acquired from the examination management server 20, and examinations to be proposed are ranked giving priority to an examination through use of an available examination device 30. By avoiding waiting for an examination through use of a busy examination device 30 in vain, a workflow can be improved in efficiency.

Alternatively, subsequent examinations may be presented with a limitation to examinations that can be executed in a subject facility. For example, examinations that can be executed in the subject facility are designated in advance on a setting window, and when presenting subsequent examinations, only the examinations that can be executed in the subject facility are presented. Examinations to be executed can be selected in accordance with the status of the facility.

A discriminator that predicts the patient's state except the name of a pathological tissue may be generated using examination data acquired utilizing the diagnosis support system. The patient's state refers to the gene mutation/appearance state, subtype, treatment responsiveness, recurrence risk score, presence/absence of recurrence, presence/absence of metastasis, an estimated metastasis site, a survival period without metastasis, an overall survival period, metastasis to a lymph node, stage classification, and the like.

Although the above description discloses an example in which HDD or a nonvolatile semiconductor memory is used as a computer-readable medium having stored therein a program for executing each type of processing, this example is not a limitation. A portable recording medium such as CD-ROM can also be applied as another computer-readable medium. A carrier wave may be applied as a medium that provides data about a program via a communication network.

Although embodiments of the present invention have been described and illustrated in detail, the disclosed embodiments are made for purposes of illustration and example only and not limitation. The scope of the present invention should be interpreted by terms of the appended claims.

What is claimed is:

1. An information processing device comprising:
a hardware processor that
acquires one or more pieces of examination data concerning a patient targeted to be diagnosed,
specifies candidates for a name of a disease by which the patient targeted to be diagnosed may be affected and a subsequent examination to be executed for determining the name of the disease of the patient targeted to be diagnosed based on the examination data as acquired, and
outputs the candidates for the name of the disease and the subsequent examination as specified.

2. The information processing device according to claim 1, wherein the hardware processor specifies the candidates for the name of the disease of the patient targeted to be diagnosed from the examination data about the patient targeted to be diagnosed using a first discriminator undergone machine learning in advance using examination data about an examination having been effective in determining the name of the disease as an input and the name of the disease of the patient undergone an examination related to the examination data as an output.

3. The information processing device according to claim 1, wherein the hardware processor specifies a subsequent examination to be executed for the patient targeted to be diagnosed from the examination data about the patient targeted to be diagnosed using a second discriminator undergone machine learning in advance using examination data about an examination having been effective in determining the name of the disease as an input and a combination of examinations having been executed for the patient undergone an examination related to the examination data and effective in determining the name of the disease as an output.

4. The information processing device according to claim 1, wherein the hardware processor specifies a subsequent examination to be executed for the patient targeted to be diagnosed using a statistical analysis based on a combination of examinations executed for determining the name of the disease, having been accumulated in advance for the candidates for the name of the disease as specified.

5. The information processing device according to claim 1, further comprising:
a memory that holds, for each patient, one or more examination items only including an examination having been executed for the patient and effective in determining the name of the disease, examination data corresponding to the examination items, and a determined diagnosis result indicating the name of the disease determined for the patient in association with each other.

6. The information processing device according to claim 1, wherein the examination data includes at least one of a medical image, a biological examination result, and clinical information.

7. The information processing device according to claim 1, wherein the candidates for the name of the disease are names of pathological tissues obtained when a diagnosis is determined.

8. The information processing device according to claim 1, wherein the subsequent examination includes at least one of an imaging modality, an imaging technique, a site to be imaged, a type of biological examination, and an examination item.

9. The information processing device according to claim 1, wherein, in a case where there are two or more candidates for the name of the disease, the hardware processor outputs a confidence rate together for each of the candidates for the name of the disease.

10. The information processing device according to claim 1, wherein the hardware processor outputs a diagnosis determination rate in a case of executing the subsequent examination.

11. The information processing device according to claim 1, wherein the hardware processor causes the candidates for the name of the disease and the subsequent examination as specified to be displayed on a display device.

12. A diagnosis support system comprising:
a hardware processor that
acquires one or more pieces of examination data concerning a patient targeted to be diagnosed,
specifies candidates for a name of a disease by which the patient targeted to be diagnosed may be affected and a subsequent examination to be executed for determining the name of the disease of the patient targeted to be diagnosed based on the examination data as acquired, and
outputs the candidates for the name of the disease and the subsequent examination as specified.

13. A non-transitory recording medium storing a computer readable program that causes a computer to:
acquire one or more pieces of examination data concerning a patient targeted to be diagnosed;
specify candidates for a name of a disease by which the patient targeted to be diagnosed may be affected and a subsequent examination to be executed for determining the name of the disease of the patient targeted to be diagnosed based on the examination data as acquired; and
output the candidates for the name of the disease and the subsequent examination as specified.
